(19) United States
(12) Patent Application Publication
(10) Pub. No.: US 2022/0417741 A1
(43) Pub. Date: Dec. 29, 2022

(54) APPLIANCE, SYSTEM AND METHOD FOR INFORMATION MANAGEMENT IN DENTISTRY

(71) Applicant: Hirsch Dynamics Holding AG, Wollerau (CH)

(72) Inventor: Markus HIRSCH, Wollerau (CH)

(73) Assignee: Hirsch Dynamics Holding AG, Wollerau (CH)

(21) Appl. No.: 17/860,307

(22) Filed: Jul. 8, 2022

Related U.S. Application Data
(63) Continuation of application No. PCT/EP2021/050025, filed on Jan. 4, 2021.
(60) Provisional application No. 62/959,261, filed on Jan. 10, 2020.

(51) Int. Cl. H04W 12/043; A61C 7/08; A61C 7/00; H04W 12/06

(52) U.S. Cl. CPC H04W 12/043; A61C 7/08; A61C 7/002; H04W 12/06; A61C 2204/005

(57) ABSTRACT

A device comprising an orthodontic or dental appliance for use with a system for information management in dentistry, a system for storing and managing dentistry data such as information about an orthodontic or dental treatment of a person using a private network and a method for information management in dentistry are described.

### CROSS REFERENCE TO RELATED APPLICATIONS

This application is a continuation of and Applicant claims priority under 35 U.S.C. § 120 of PCT Application No. PCT/EP2021/050025 filed on Jan. 4, 2021, which claims priority under 35 U.S.C. § 119(e) from U.S. Provisional Patent Application Ser. No. 62/959,261 filed Jan. 10, 2020, the disclosures of each of which are hereby incorporated by reference. The International Application under PCT article 21(2) was published in English.

### BACKGROUND OF THE INVENTION

### 1. Field of the Invention

In one aspect, the present invention relates to a device comprising an orthodontic or dental appliance for use with a private network.

In another aspect, the present invention relates to a system for storing and managing information about an orthodontic or dental treatment of a person (sometimes called “patient”) using a private network, in particular a network using distributed ledger technology and a method for information management in dentistry.

In yet another aspect, the present invention relates to a method for information management in dentistry.

### 2. Description of he Related Art

A dentition of a patient is the arrangement of teeth on the lower jaw (mandible) or the upper jaw (maxilla). An occlusal state (or bite pattern) is defined by a given dentition on the mandible, a given dentition on the maxilla and the relative arrangement of mandible and maxilla (jaw alignment).

Abnormal arrangement of teeth in a dentition can lead to misaligned occlusal states (malocclusion or misalignment). The presence of a misaligned state is also called an orthodontic condition.

Treating a certain orthodontic condition via an alignment of a malocclusion and/or a misalignment, respectively, by transferring a first occlusal state into a second occlusal state (that in general differs from the first occlusal state and can be called target state) can be of medical and/or aesthetic interest.

It is possible to balance out jaw misalignments and/or malocclusions for a specific orthodontic condition with respect to medical reasons and/or realign an occlusal state for the sake of—particularly subjective—optical advantages with respect to a patient's cosmetic correction (generated, e.g., by an orthodontic appliance like an aligner).

In general, an aesthetic dental condition can be seen as an orthodontic condition, even if there is no medical necessity present. It can also be desired to maintain a specific occlusal state, e.g., via a permanent or removable lingual retainer.

The term “dentistry” is understood to encompass all practices related to the oral cavity, including dental and orthodontic practices.

In the present disclosure the term “dental” is used to mean practices performed by a dentist or dental surgeon such as restorative practices (fillings, crowns, bridges), prosthetic practices (dentures), endodontic (root canal) therapy, periodontal (gum) therapy, and extraction of teeth, as well as performing examinations, radiographs (x-rays), and diagnosis.

The term “orthodontic” is used to mean practices performed by an orthodontist such as the diagnosis, prevention, and correction of malpositioned teeth and jaws, and misaligned bite patterns.

The term “orthodontic appliance” refers to any device which is put in place by orthodontists to gradually reposition teeth to a desired alignment or to retain a desired alignment. There are “fixed orthodontic appliances” (e.g., bands, wires, brackets, lingual retainers) that are bonded to the teeth and are not supposed to be removed by the patient, and “removable orthodontic appliances” which can be removed by a patient (e.g., for cleaning), such as aligners.

The term “dental appliance” refers to any device which is put in place by dentists such as a dental implant, a dental bridge or dental crown.

A system and method for orthodontic care management is described in US 2020/0066391 A1. The use of blockchain technology is contemplated, however, it remains unclear how a patient would gain access to a blockchain in a secure but easy way.

Another system for managing medical records using a multi-dimensional cryptographically secured datastore is described in US 2020/0160947 A1. It is not discussed how a patient could access the datastore in a secure but easy way.

A method of reducing counterfeit medicaments by providing a medicament carrier which is to be placed in an oral cavity of a patient having a medicament disposed therein or thereon is described in WO 2019/160760 A1. The medicament carrier can be used in a blockchain system.

What is needed is a device, a system and a method which facilitate secure access to a private network for a person without the need of the person having more than average IT skills or the person having to provide proof-of-work such as mining, and to allow temper-evident storage of data to be stored in the private network.

### SUMMARY OF THE INVENTION

It is an object of the invention to provide a device which facilitates access to a private network for a person without the need of the person having more than average IT skills or the person having to provide proof-of-work such as mining.

It is another object of the invention to provide a system for storing and managing information about an orthodontic or dental treatment of a person using a private network with a secure but easy way to access the private network.

It is another object of the invention to provide a method for information management in dentistry, e.g., storing and managing information about an orthodontic or dental treatment of a person using a private network with a secure but easy way to access the private network.

It is yet another object of the invention to provide a computer program, a computer-readable, in particular non-transitory, medium and a data carrier signal.

Still other objects and advantages of the invention will in part be obvious and will in part be apparent from the specification and drawings.

One object of the disclosure relates to a device which comprises:
an orthodontic or dental appliance
a (preferably machine-readable) code which is arranged on or in the appliance, wherein the code represents at least:
   a key to access a private network, preferably a permissioned distributed ledger network
   access to data to be stored in the private network.

It is an advantage of such a device and system that a patient can, in an easy-to-use but secure way, access the private network using the orthodontic or dental appliance as a key. However, the appliance not only serves as a key but also as an information carrier for information that is to be added to the private network such as information specific to the appliance and/or to the patient and/or to a treatment history of the patient.

By way of example, the key to access the private network can be a private key of an asymmetric key pair consisting of a public key and a private key. The public key can be stored in the private network, e.g., in a blockchain, while the corresponding private key is stored on or in the orthodontic or dental appliance. Any part of the private network such as a network node is able to verify the identity of an appliance by accessing its public key that lies in the network and challenging the appliance. Other secure identification methods which are known in the art could be used.

By way of example, access to data can mean that the data itself is given by way of the code, i.e., the code directly encodes the data to be stored. In addition or alternatively, access to data can mean that the code could represent a link to the data which is to be stored, e.g., a link which can be used by a component of the private network, such as a node of the private network, to access data which is stored on a database externally to the private network and to import this data into the private network.

Another object of the disclosure relates to a system for storing and managing information about an orthodontic or dental treatment of a person, which comprises:
a private network, with at least one authorizing device which, upon receiving an access query, permits or refuses access to the private network based on a key provided with the access query
an input device configured to read the code provided with at least one device of at least one embodiment of a device according to the invention and to make the code available to the at least one authorizing device.

Another object of the disclosure relates to a method for information management in dentistry comprising at least the following steps:

providing a private network for information management in dentistry, preferably a permissioned distributed ledger network
providing a code which represents at least:
   a key to access the private network
   access to data to be stored in the private network
manufacturing an orthodontic or dental appliance having the code arranged on or in the appliance
using the key provided with the code to access the private network and to store the data provided with the code in the private network

Yet other objects of the disclosure relate to a computer program, a computer-readable, in particular non-transitory, medium and a data carrier signal.

Embodiments of the invention are defined in the dependent claims.

### DESCRIPTION OF EMBODIMENTS

A dental appliance can be any device which is put in place by dentists such as a dental implant, a dental bridge or dental crown.

An orthodontic appliance can be any device which is put in place by orthodontists to gradually reposition teeth to a desired alignment or to retain a desired alignment such as “fixed orthodontic appliances” (e.g., bands, wires, brackets, lingual retainers) that are bonded to the teeth and are not supposed to be removed by the patient, and removable orthodontic appliances” which can be removed by a patient (e.g., for cleaning), such as aligners.

Information management in dentistry means the acquisition dentistry data from different sources (e.g., a dentist or orthodontist, a manufacturer of appliances, a patient, . . . ), the custodianship and the distribution of that data to those who need it (e.g., a dentist or orthodontist, a manufacturer of appliances, a patient, . . . ), and archiving this data in a private and temper-evident manner.

Dentistry data to be stored in the private network can be or can comprise any type of information relating to dentistry, in particular information specific to the appliance and/or to a person for which the appliance is intended and/or to a treatment history of a person for which the appliance is intended.

By way of example, information specific to the appliance can be: identification of appliance, in particular with respect to a patient, data relating to manufacture of appliance (such as identification of manufacturer, date of manufacture, location of manufacture, production traceability from raw material to the finished product, .), data relating to full integration of value chain traceability for the appliance, number of appliance in treatment plan which needs several appliances, usage statistics of appliance (e.g., how long was an aligner worn by the patient), etc.

By way of example, information specific to a person for which the appliance is intended can be: identification of person, medical data of person, orthodontic treatment plan, identification of dentist and/or orthodontist, information relating to patient data management/administration, etc.

By way of example, information specific to a treatment history of a person for which the appliance is intended can be: information regarding dentition and/or malocclusion, information about past dental or orthodontic treatments, current treatment plan including, e.g., which aligner is to be used at a present time, etc.

Although it is preferred that the code arranged on or in the appliance is machine-readable it can be envisioned that a person is asked to manually enter the code in an interface of the private network.

If the code is to be arranged inside the appliance it is advantageous if the appliance is transparent at least to an extent that the code can be read by a machine or a person. If the appliance is not completely transparent, it could be envisioned to add a transparent window in the region of the code.

In some embodiments of the device at least one of the following groups is provided as carrier for the (preferably machine-readable) code:
a chip, preferably an RAD-chip
a code which is embedded inside of and/or formed by a material of the appliance (e.g., by lasering, printing or etching), preferably in the form of a bar code or a two-dimensional code.

In these embodiments it can be provided that
a receiving device for receiving information and/or a sending device for sending information, and
an electronic storage for storing information received by the receiving device and/or to be sent by the sending device

are arranged on or in the appliance.

By way of example the receiving device could be used to update information present in the code.

By way of example the sending device could be used to actively communicate with an interface of the system. In other embodiments, communication with the system is passive, i.e., the system must access the information represented by the code.

By way of example the electronic storage could be used to store a digital version of the complete code and/or part of the code only. In the latter case the part of the code could relate to the key to access the private network or the information other than the key.

In some of these embodiments the receiving device is connected to or formed as a sensor to receive information about ambient conditions to which the at least one appliance is exposed. By way of example the sensor could be used to determine when an appliance is carried inside the mouth of a patient (e.g., by measuring temperature and/or humidity).

In some of these embodiments the receiving device and/or the sending device are or is connected to or formed as an antenna for wirelessly receiving and/or sending information.

There can be provided a receptacle comprising at least:
a cavity adapted to store a device of at least one of the devices according to at least one of the embodiments discussed above
a communication device for sending information to and/or receiving information from the appliance.

In some embodiments the communication device can comprise at least one of the groups of: an NFC communication device, an optical scanner, a magnetic scanner,

In some embodiments the receptacle can be used as an interface for facilitating communication between the device and the system.

In some embodiments the receptacle can be used to track how long an appliance is arranged inside the receptacle (and is therefore not worn by the patient) .

In some embodiments of the invention the private network comprises at least one centralized database. In this embodiment the authorizing device can be a portal which accepts the key which is arranged on or in the appliance as a valid login request to the database.

However, in preferred embodiments of the invention the private network is a permissioned distributed ledger etwork, with a plurality of nodes and the at least one authorizing device is formed by at least one authorizing node which, upon receiving an access query, permits or refuses access to nodes of the distributed ledger network based on the key provided with the access query.

A distributed ledger network is a decentralized, peer-to-peer network in which a plurality of computing devices (also called nodes), store transactional data in a verifiable, tamper-evident manner. Distributed-ledger networks can use a variety of cryptographic techniques to submit and store data which can be verified, based on agreed-upon algorithms and a consensus of validity among the participating nodes. Each node replicates and saves an identical copy of the ledger and updates itself independently. Any consensus protocol known in the art can be used to specify the circumstances under which data can be added to the distributed ledger network, e.g., when data is inputted to the ledger, i.e., a ledger update happens, each node constructs the new transaction, and then the nodes vote on which copy is correct. Once a consensus has been determined, all the other nodes update themselves with the new, correct copy of the ledger. Alternative consensus algorithms are proof-of-work and proof-of-stake.

In preferred embodiments it is considered sufficient if a key is provided upon which access to the private network is granted, i.e., in these embodiments no further consent is necessary.

In a permissioned distributed ledger network only participants of a selected group of participants (such as dentists, orthodontists, patients, manufacturers of appliances, . . . ) is allowed access to the distributed ledger network by use of keys. The range of usable consensus algorithms is broader than in permissionless distributed ledger networks since only the participants of the selected group of participants can access the network and the risk of impersonation attacks does not exist.

In some embodiments the distributed ledger network comprises at least one plurality of nodes which are configured to implement a blockchain protocol and wherein, upon permission by the at least one authorizing node to access a node of the distributed ledger network, a block adding transaction is carried out by which a block is added as a subsequent block to a preceding block, the block which is added as a subsequent block comprising at least:
a cryptographic link to the preceding block
the data to be stored in the private network, such as: information specific to the orthodontic appliance and/or to a person for which the appliance is intended and/or to a treatment history of a person for which the appliance is intended, which information was provided with the at least one appliance.

In some embodiments an interface can be provided which can be used to facilitate communication between the code provided with the appliance and the system.

By way of example, the interface could be designed in the form of an app loaded onto a smartphone. The smartphone could use NFC-technology to communicate with a chip carrying the code and/or it could use its camera to scan a code provided by way of a barcode or two-dimensional code. The smartphone could use access to a communication network such as the internet to communicate with the system.

By way of another example, the interface could be designed in the form of a website which can be accessed by a user and which by way of a suitable computer interface, e.g., a scanner or camera, could access the code of the device.

By way of yet another example, the interface could be designed in the form of a receptacle as described above.

By way of example the complete appliance, or any part thereof, can be formed of ceramic, composite, or metal and is preferably translucent, opaque or fully transparent ceramic or composite material (e.g., aluminum oxide or zirconium oxide ceramics).

In some embodiments of the method a permissioned distributed ledger network is used as the private network.

In these embodiments it can be provided that a blockchain protocol is used to store the data for which access is provided with the code in blocks of a blockchain.

### BRIEF DESCRIPTION OF THE DRAWINGS

Other objects and features of the invention will become apparent from the following detailed description considered in connection with the accompanying drawings. It is to be understood, however, that the drawings are designed as an illustration only and not as a definition of the limits of the invention.

In the drawings,

FIG. 1 shows a schematic view of a device according to an embodiment of the invention;

FIG. 2 shows a schematic view of a chip for use with a device according to an embodiment of the invention;

FIG. 3 shows a schematic view of an interaction of an interface with a chip;

FIG. 4 shows a schematic view of a presentation of information using interfaces;

FIGS. 5A-5C show schematic views of a receptacle with a device according to an embodiment of the invention;

FIG. 6 shows a first embodiment of a system according to the invention;

FIG. 7 shows a schematic view of a second embodiment of a system according to the invention; and

FIG. 8 shows a schematic view of details regarding the second embodiment of the system according to the invention.

### DETAILED DESCRIPTION OF PREFERRED EMBODIMENTS

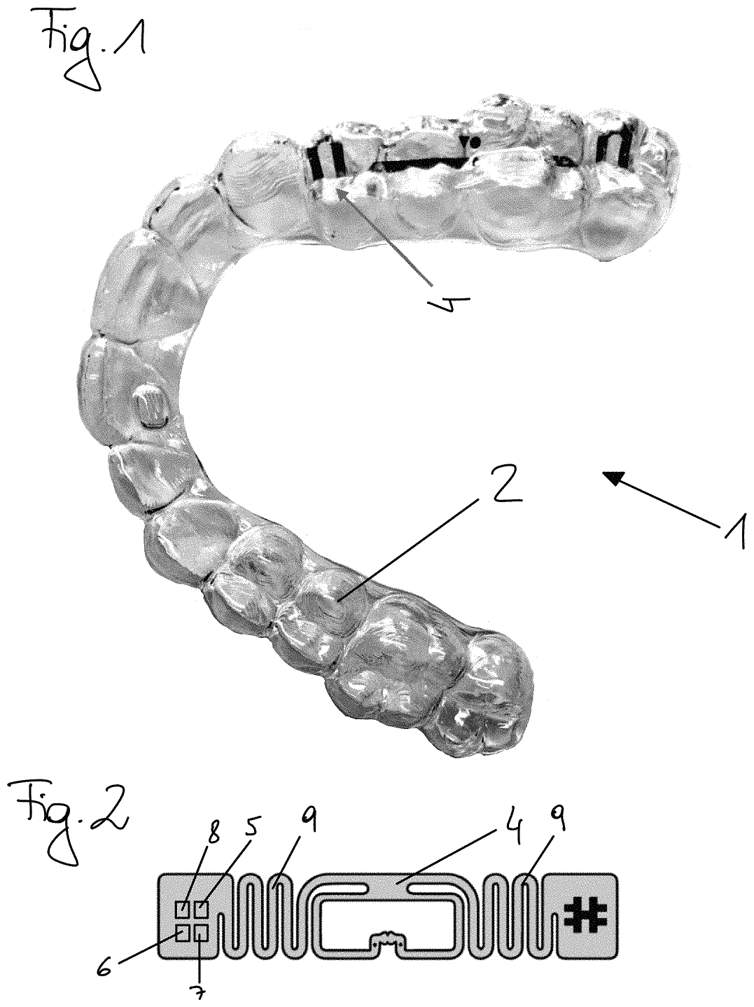

FIG. 1 shows a device 1 comprising:
an (orthodontic) appliance 2, in this example an aligner
   a machine-readable code 3 which is carried by a chip 4 which is arranged on the appliance 2, wherein the code 3 represents:
      a key to access a private network
      information specific to the appliance 2 and to a person for which the appliance 2 is intended and to a treatment history of a person for which the appliance 2 is intended.

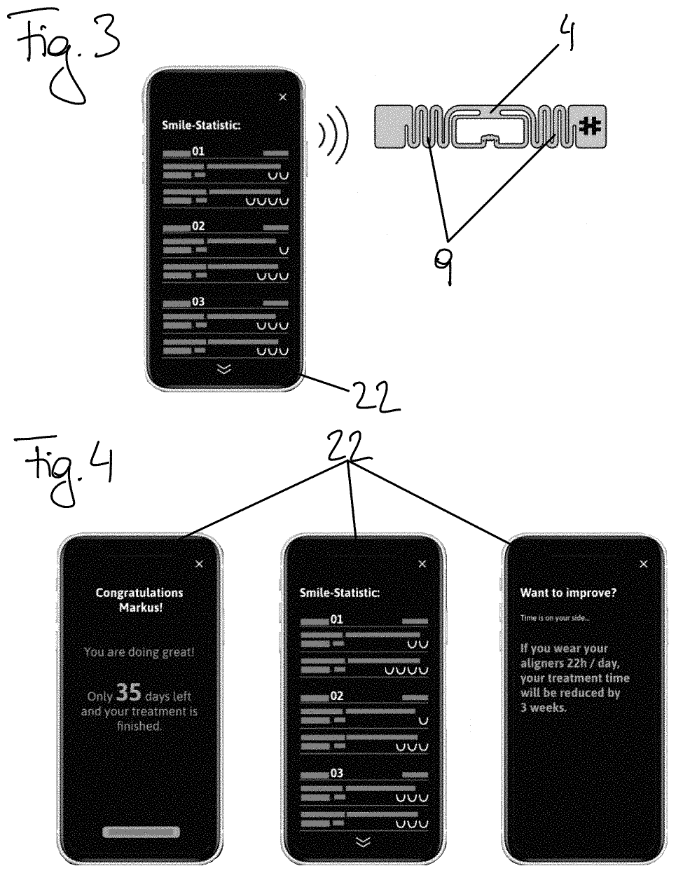

FIG. 2 shows an example of the chip 4 in more detail (but in a schematic way—the components shown need not be present as distinct physical elements but could be present by way of a single or several integrated circuit(s)). The chip 4, in this example, has a receiving device 5, a sending device 6, an electronic storage 7, a sensor 8, and an antenna 9.

FIG. 3 shows an interface 22 (here in the form of an app loaded onto a smartphone) which can be used to communicate with the chip 4 (the appliance 2 on or in which the chip 4 is placed is not shown).

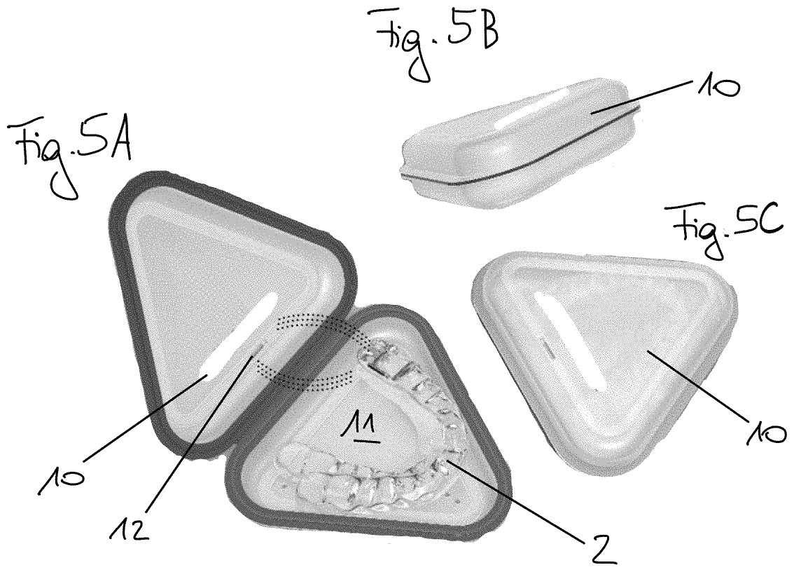

FIG. 4 shows that the interface 22 shown in FIG. 3 can be used to present different kinds of information.

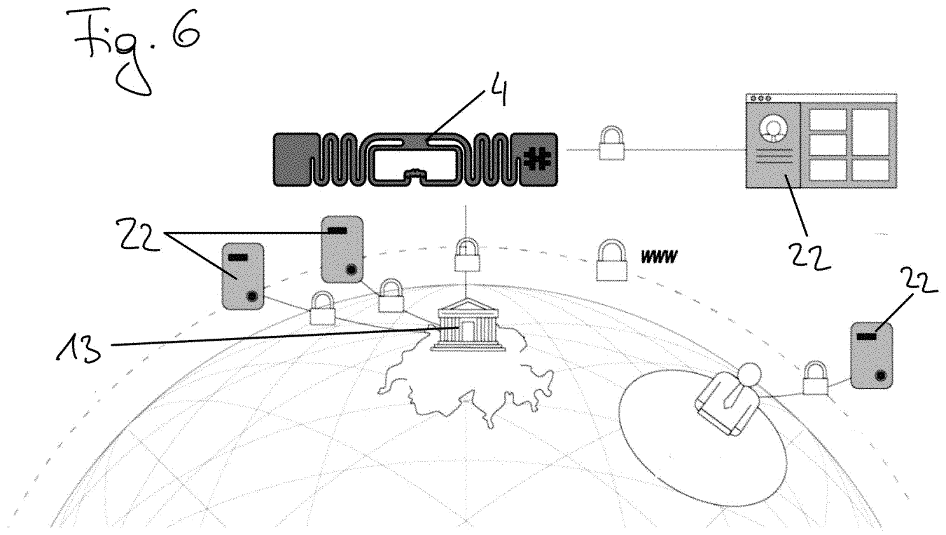

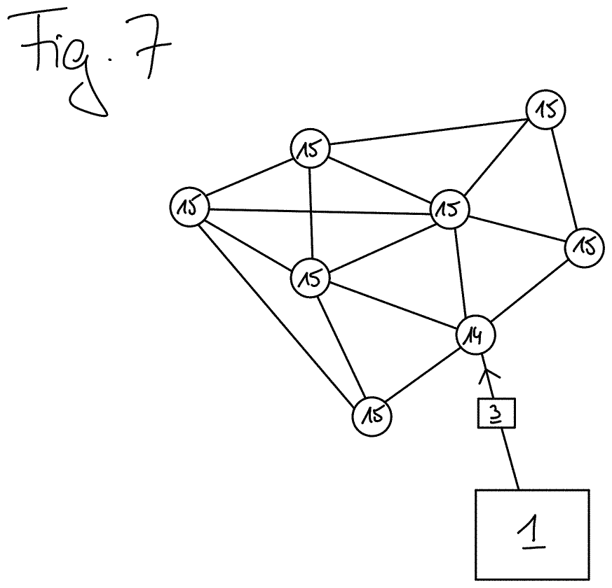

FIGS. 5A-5C show an arrangement with a receptacle 10 comprising:
a cavity 11 adapted to store the device 1 shown in FIG. 1
a communication device 12 for sending information to and/or receiving information from the appliance 2 of the device 1.

FIG. 6 shows a first embodiment of the system in which the private network comprises at least one centralized database 13 which can be accessed via interfaces 22 which provide the code 3 stored on the chip 4 of the device 1 as key to an authorizing device (e.g., any kind of interface) of the database 13.

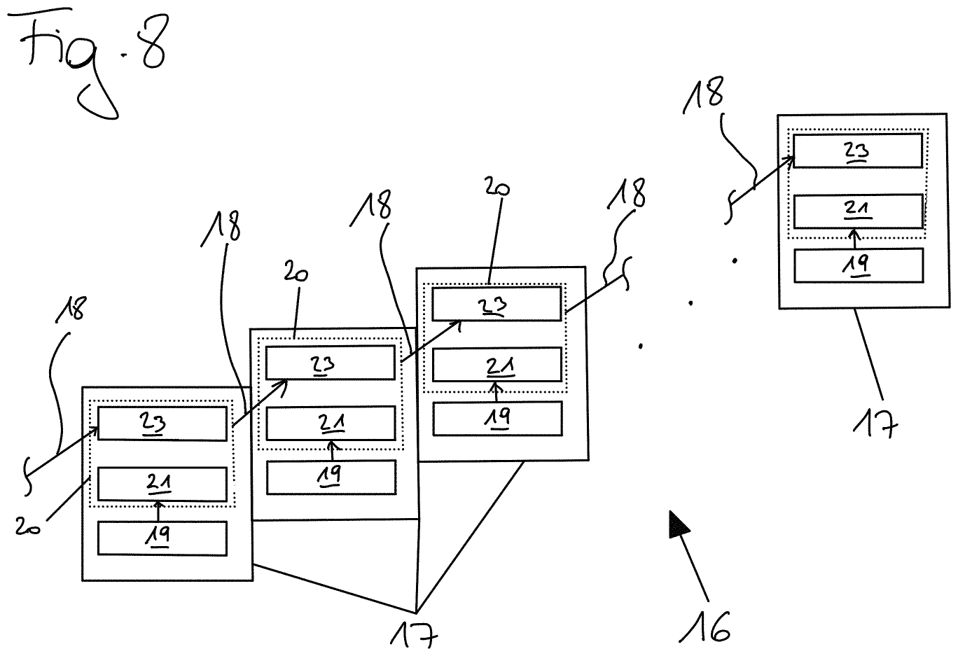

FIG. 7 shows a second embodiment of the system in which the private network is a permissioned distributed ledger network, with a plurality of nodes 15 and at least one authorizing device which is formed by at least one authorizing node 14 which, upon receiving an access query carrying the code 3 (which incorporates a key for the system), permits access to the nodes 15 of the distributed ledger etwork.

FIG. 8 shows a detail of the system of FIG. 7 in which the distributed ledger network comprises at least one plurality of nodes 15 which are configured to implement a blockchain protocol

Upon permission by the at least one authorizing node 14 to access a node 15 of the distributed ledger network, a block adding transaction is carried out by which a block 17 is added as a subsequent block 17 to a preceding block 17 of a blockchain 16.

The block 17 which is added as a subsequent block 17 comprises at least:
a header 20 comprising at least a block ID, a cryptographic representation of transaction data of the present block 17, such as a Merkle root 21 of the transaction data and a cryptographic link 18 to the preceding block 17 (here: hash value 23 of header 20 of preceding block 17), and, possibly, further parameters as known in the art, such as a timestamp, and
transaction data of the present block 17 in the form of the information 19 specific to the appliance 2 and/or to a person for which the appliance 2 is intended and/or to a treatment history of a person for which the appliance 2 is intended, which information was provided with the at least one appliance 2.

Although only a few embodiments of the present invention have been shown and described, it is to be understood that many changes and modifications may be made thereunto without departing from the spirit and scope of the invention.

### REFERENCE SIGNS LIST

1 device
2 appliance
3 code
4 chip
5 receiving device
6 sending device
7 electronic storage
8 sensor
9 antenna
10 receptacle
11 cavity
12 communication device of receptacle
13 database
14 authorizing node
15 nodes of private network
16 blockchain
17 block of blockchain
18 cryptographic link
19 information
20 header
21 Merkle root
22 interface
23 hash value of previous block

What is claimed is:

1. A device, comprising:
an orthodontic or dental appliance, and
a, preferably machine-readable, code which is arranged on or in the appliance, wherein the code represents at least:
a key to access a private network, preferably a permissioned distributed ledger network
access to dentistry data to be stored in the private network.

2. The device of claim 1, wherein the dentistry data to be stored in the private network is or comprises information as defined by at least one of the following groups: information specific to the appliance, information specific to a person for which the appliance is intended, information specific to a treatment history of a person for which the appliance is intended.

3. The device of claim 1, wherein at least one of the following groups is provided as carrier for the code:
a chip, preferably an RFID-chip, and
a code which is embedded inside of and/or formed by a material of the appliance, preferably in the form of a bar code or a two-dimensional code.

4. The device of claim 1, wherein
a receiving device for receiving information and/or a sending device for sending information, and
an electronic storage for storing information received by the receiving device and/or to be sent by the sending device
are arranged on or in the appliance.

5. The device of claim 1, wherein the receiving device is connected to or formed as a sensor to receive information about ambient conditions to which the at least one appliance is exposed.

6. The device of claim 4, wherein the receiving device and/or the sending device are or is connected to or formed as an antenna for wirelessly receiving and/or sending information.

7. A receptacle comprising at least:
a cavity adapted to store a device of claim 1, and
a communication device for sending information to and/or receiving information from the appliance.

8. An arrangement comprising at least:
a receptacle according to claim 7, and
a device of at least one of claim 1 to claim 6 stored in the cavity of the receptacle.

9. A system for storing and managing information about an orthodontic or dental treatment of a person, comprising:
a private network, with at least one authorizing device which, upon receiving an access query, permits or refuses access to the private network based on a key provided with the access query, and
an input device configured to read the code provided with at least one device of claim 1 and to make at least the key comprised in the code available to the at least one authorizing device.

10. The system according to claim 9, wherein the private network comprises at least one centralized database.

11. The system according to claim 9, wherein the private network is a permissioned distributed ledger network, with a plurality of nodes and the at least one authorizing device is formed by at least one authorizing node which, upon receiving an access query, permits or refuses access to nodes of the distributed ledger network based on the key provided with the access query.

12. The system according to claim 11, wherein the distributed ledger network comprises at least one plurality of nodes which are configured to implement a blockchain protocol and wherein, upon permission by the at least one authorizing node to access a node of the distributed ledger network, a block adding transaction is carried out by which a block is added as a subsequent block to a preceding block of a blockchain.

13. The system according to claim 12, wherein the block which is added as a subsequent block comprises at least:
a header comprising at least a block ID, a cryptographic representation of transaction data of the present block, and a cryptographic link to the preceding block 17, and
transaction data of the present block in the form of the dentistry data.

14. A method for information management in dentistry, comprising at east the following steps:
providing a private network for information management in dentistry,
preferably a permissioned distributed ledger network
providing a code which represents at least:
a key to access the private network
access to dentistry data to be stored in the private network
manufacturing an orthodontic or dental appliance having the code arranged on or in the appliance, and
using the key provided with the code to access the private network and to store the data for which access is provided with the code in the private network.

15. The method of claim 14, wherein a permissioned distributed ledger network is used as the private network.

16. The method of claim 14, wherein a blockchain protocol is used to store the dentistry data for which access is provided with the code in blocks of a blockchain.

17. A computer program which, when the program is executed by a computer, causes the computer to be configured as an authorizing device of a system according to claim 9.

18. A computer-readable, in particular non-transitory, medium comprising instructions which, when executed by a computer, causes the computer to be configured as an authorizing device of a system according to claim 9.

19. A data carrier signal carrying the computer program of claim 17.
